(19) United States
(12) Patent Application Publication
(10) Pub. No.: US 2024/0041603 A1
(43) Pub. Date: Feb. 8, 2024

(54) LEAFLET RESTRAINTS

(71) Applicant: SV SWISSVORTEX AG, Zurich (CH)

(72) Inventors: Andrea GUIDOTTI, Zurich (CH); David ZARBATANY, Laguna Niguel, CA (US); Pietro GOZZOLI, Zurich (CH)

(73) Assignee: SV SWISSVORTEX AG, Zurich (CH)

(21) Appl. No.: 18/266,510

(22) PCT Filed: Dec. 9, 2021
(86) PCT No.: PCT/IB2021/061479

§ 371 Date: Jun. 9, 2023

Related U.S. Application Data
(60) Provisional application No. 63/123,530, filed on Dec. 10, 2020.

(51) Int. Cl. A61F 2/24; A61B 17/04

(52) U.S. Cl. CPC A61F 2/2463; A61B 17/0401; A61B 17/0467; A61B 2017/0409; A61B 2017/0427

(57) ABSTRACT

A leaflet restraint (20) includes a non-blood-occlusive net (30), which is configured to at least partially cover an atrial surface (32) of a single native cardiac leaflet (34) of a native atrioventricular valve (36) of a heart (38). Two or more tissue-penetrating annulus anchors (40) are fixed to an annular side (42) of the net (30), and are configured to be anchored to an annulus (44) of the native atrioventricular valve (36) along or within 1 cm of a portion of the annulus (44) to which the single native cardiac leaflet (34) attaches. A ventricular anchor (50) is configured to be anchored to a ventricle (54) of the heart (58) so as to anchor a ventricular end (52) of the net (30) to the ventricle (54). The leaflet restraint (20) is configured, when anchored in place at least partially covering the atrial surface (32) of the single native cardiac leaflet (34), to limit prolapse of the single native cardiac leaflet (34) into an atrium (56) of the heart (38). Other embodiments are also described.

### CROSS-REFERENCE TO RELATED APPLICATIONS

The present patent application claims priority from U.S. Provisional Application 63/123,530, filed Dec. 10, 2020, which is assigned to the assignee of the present application and incorporated herein by reference.

### FIELD OF THE INVENTION

The present invention relates generally to techniques for treating atrioventricular valve degeneration including prolapse and flail.

### BACKGROUND OF THE INVENTION

Mitral valve degenerative disease typically manifests in the form of valve prolapse or valve flail. Mitral valve prolapse is the bulging of one or both of the mitral valve leaflets into the left atrium during the contraction of the heart. Mitral valve flail is the movement of the leaflet's free edge into the atrium, above the valve annular plane, typically due to chordae rupture. Mitral valve degenerative disease sometimes causes mitral regurgitation, which may lead to heart failure, atrial fibrillation, and/or pulmonary hypertension.

### SUMMARY OF THE INVENTION

Embodiments of the present invention provide a leaflet restraint that is configured to limit prolapse of the native leaflet into an atrium of the heart. The leaflet restraint is not configured to function as a prosthetic leaflet or valve, and thus does not occlude blood flow therethrough.

The leaflet restraint comprises a non-blood-occlusive net, which is configured to at least partially cover an atrial surface of a single native cardiac leaflet of a native atrioventricular valve of a heart. Two or more tissue-penetrating annulus anchors are fixed to an annular side of the net, and are configured to be anchored to an annulus of the native atrioventricular valve along or within 1 cm of a portion of the annulus to which the single native cardiac leaflet attaches. A ventricular anchor is configured to be anchored to a ventricle of the heart so as to anchor a ventricular end of the net to the ventricle. Typically, the leaflet restraint does not comprise any annulus anchors that are configured to be anchored to the annulus other than along or within 1 cm of the portion of the annulus to which the single native cardiac leaflet attaches. The leaflet restraint is configured, when anchored in place at least partially covering the atrial surface of the single native cardiac leaflet, to limit prolapse of the single native cardiac leaflet into an atrium of the heart.

There is therefore provided, in accordance with an application of the present invention, a leaflet restraint including:
a non-blood-occlusive net, which is configured to at least partially cover an atrial surface of a single native cardiac leaflet of a native atrioventricular valve of a heart;
two or more tissue-penetrating annulus anchors, which are fixed to an annular side of the net, and which are configured to be anchored to an annulus of the native atrioventricular valve along or within 1 cm of a portion of the annulus to which the single native cardiac leaflet attaches; and
a ventricular anchor, which is configured to be anchored to a ventricle of the heart so as to anchor a ventricular end of the net to the ventricle,
wherein the leaflet restraint does not include any annulus anchors that are configured to be anchored to the annulus other than along or within 1 cm of the portion of the annulus to which the single native cardiac leaflet attaches, and
wherein the leaflet restraint is configured, when anchored in place at least partially covering the atrial surface of the single native cardiac leaflet, to limit prolapse of the single native cardiac leaflet into an atrium of the heart.

For some applications, the ventricular anchor is configured to be anchored to a ventricular site within the ventricle, the site selected from the group of sites consisting of: a papillary muscle, a chorda tendinea, a free edge of another native cardiac leaflet, a ventricular wall, and ventricular trabeculae.

For some applications, the ventricular anchor is configured to be anchored to the ventricle so as to directly anchor the ventricular end of the net to the ventricle.

For some applications, the leaflet restraint further includes a tether that is fixed to the ventricular end of the net, and the ventricular anchor is configured to anchor the tether to the ventricle so as to anchor the ventricular end of the net to the ventricle.

For some applications, the net is configured to cover only a portion of the atrial surface of the single native cardiac leaflet, the portion including an annular edge of the single native cardiac leaflet.

For some applications, the net is configured to cover (a) a portion of the atrial surface of the single native cardiac leaflet, the portion including an annular edge of the single native cardiac leaflet, and (b) a free ventricular edge of the single native cardiac leaflet.

For some applications, the net defines a plurality of openings and the net, when in a fully-expanded planar configuration, has an area, including the plurality of openings, of at least 100 mm2.

For some applications:
the net includes a plurality of flexible elongate members arranged so as to define a plurality of openings, and
the net, when in a fully-expanded planar configuration, has a material surface area ratio of 5%-80%, the material surface area ratio being the ratio of the area of material of the flexible elongate members to an area of the net, including the plurality of openings and the material of the flexible elongate members.

For some applications, the net, when in a fully-expanded planar configuration, includes a triangular portion.

For some applications, the net, when in a fully-expanded planar configuration, is triangular.

For some applications, the net, when in a fully-expanded planar configuration, includes a rectangular portion.

For some applications, the net, when in a fully-expanded planar configuration, is rectangular.

For some applications, the net includes a plurality of flexible elongate members arranged at least partially as a grid when in a fully-expanded planar configuration.

For some applications, the net includes a plurality of flexible elongate members arranged at least partially in a spider-web configuration when in a fully-expanded planar configuration.

For some applications, the leaflet restraint further includes an annular support that extends along at least a portion of the annular side of the net, in order to maintain a length of the annular side of the net.

For some applications, the annular support is defined by a discrete element fixed to the at least a portion of the net.

For some applications, the discrete element includes a rod.

For some applications:
the net includes a plurality of flexible elongate members,
the annular support is defined by the at least a portion of the annular side of the net, and
the flexible elongate members of the at least a portion of the annular side of the net are arranged so to provide the annular support with greater rigidity than other portions of the net.

For some applications, the flexible elongate members of the at least a portion of the annular side of the net are woven so to provide the annular support with greater rigidity than other portions of the net.

For some applications, the flexible elongate members of the at least a portion of the annular side of the net are rolled in a scroll-shaped arrangement so to provide the annular support with the greater rigidity than other portions of the net.

For some applications, the two or more tissue-penetrating annulus anchors are fixed with respect to the annular support.

For some applications, the two or more tissue-penetrating annulus anchors are fixed directly to the annular support.

For some applications, the net defines a plurality of openings having an average opening area of at least 4 mm2, when the net is in a fully-expanded planar configuration.

For some applications, the average opening area is at least 10 mm2.

For some applications, the net defines a plurality of openings, and two or more of the openings have respective opening areas different from one another, when the net is in a fully-expanded planar configuration.

For some applications, three or more of the openings have respective opening areas different from one another, when the net is in the fully-expanded planar configuration.

For some applications, five or more of the openings have respective opening areas different from one another, when the net is in the fully-expanded planar configuration.

For some applications, the opening area of a first one of the two or more of the openings equals at least 110% of the opening area of a second one of the two or more of the openings, when the net is in the fully-expanded planar configuration.

For some applications, the opening area of the first opening equals at least 120% of the opening area of the second opening, when the net is in the fully-expanded planar configuration.

For some applications, the opening areas of the plurality of openings vary along the net between the annular side and the ventricular end of the net.

For some applications, the opening areas of the plurality of openings decrease along the net between the annular side and the ventricular end of the net.

For some applications, the opening areas of the plurality of openings increase along the net between the annular side and the ventricular end of the net.

For some applications, an average of the opening areas of the plurality of openings on an annular half of the net is different from an average of the opening areas of the plurality of openings on a ventricular half of the net.

For some applications, the average of the opening areas of the plurality of openings on the annular half of the net is greater than the average of the opening areas of the plurality of openings on the ventricular half of the net.

For some applications, the average of the opening areas of the plurality of openings on the annular half of the net is less than the average of the opening areas of the plurality of openings on the ventricular half of the net.

For some applications, a leaflet-restraint system is provided that includes the leaflet restraint,
the leaflet restraint is a first leaflet restraint, the non-blood-occlusive net is a first non-blood-occlusive net, the two or more tissue-penetrating annulus anchors are two or more first tissue-penetrating annulus anchors, and the ventricular anchor is a first ventricular anchor,
the leaflet-restraint system further includes a second leaflet restraint, which includes:
   a second non-blood-occlusive net, which is configured to at least partially cover the atrial surface of the single native cardiac leaflet;
   two or more second tissue-penetrating annulus anchors, which are fixed to an annular side of the second net, and which are configured to be anchored to the annulus of the native atrioventricular valve along or within 1 cm of a portion of the annulus to which the single native cardiac leaflet attaches; and
   a second ventricular anchor, which is configured to be anchored to the ventricle so as to anchor a ventricular end of the second net to the ventricle, and
the first and the second leaflet restraints are configured, when anchored in place at least partially covering the atrial surface of the single native cardiac leaflet, to limit prolapse of the single native cardiac leaflet into the atrium.

There is further provided, in accordance with an application of the present invention, a method for treating a single native cardiac leaflet of a native atrioventricular valve of a heart, the method including:
anchoring two or more tissue-penetrating annulus anchors of a leaflet restraint to an annulus of the native atrioventricular valve along or within 1 cm of a portion of the annulus to which the single native cardiac leaflet attaches;
at least partially covering an atrial surface of the single native cardiac leaflet with a non-blood-occlusive net of the native leaflet restraint, wherein the tissue-penetrating annulus anchors are fixed to an annular side of the net; and
anchoring a ventricular anchor of the leaflet restraint to a ventricle of the heart so as to anchor a ventricular end of the net to the ventricle, such that the leaflet restraint limits prolapse of the single native cardiac leaflet into an atrium of the heart,
wherein the method does not include anchoring any annulus anchors to the annulus other than along or within 1 cm of the portion of the annulus to which the single native cardiac leaflet attaches.

For some applications, anchoring the ventricular anchor includes anchoring the ventricular anchor at an anchoring site within the ventricle that sets a desired angle of a best-fit plane defined by the net with respect to a plane of the annulus, in order to set a level of restraint of the single native cardiac leaflet.

For some applications, anchoring the ventricular anchor to the ventricle includes anchoring the ventricular anchor to a ventricular site within the ventricle, the site selected from the group of sites consisting of: a papillary muscle, a chorda tendinea, a free edge of another single native cardiac leaflet, a ventricular wall, and ventricular trabeculae.

For some applications, the native leaflet restraint further includes an annular support that extends along at least a portion of the annular side of the net, in order to maintain a length of the annular side of the net.

For some applications, the annular support is defined by a discrete element fixed to the at least a portion of the net.

For some applications, the discrete element includes a rod.

For some applications:
the net includes a plurality of flexible elongate members,
the annular support is defined by the at least a portion of the annular side of the net, and
the flexible elongate members of the at least a portion of the annular side of the net are arranged so to provide the annular support with greater rigidity than other portions of the net.

For some applications, the flexible elongate members of the at least a portion of the annular side of the net are woven so to provide the annular support with greater rigidity than other portions of the net.

For some applications, the flexible elongate members of the at least a portion of the annular side of the net are rolled in a scroll-shaped arrangement so to provide the annular support with the greater rigidity than other portions of the net.

For some applications, the two or more tissue-penetrating annulus anchors are fixed with respect to the annular support.

For some applications, the two or more tissue-penetrating annulus anchors are fixed directly to the annular support.

For some applications, anchoring the ventricular anchor to the ventricle including using the ventricular anchor to directly anchor the ventricular end of the net to the ventricle.

For some applications, the leaflet restraint further includes a tether that is fixed to the ventricular end of the net, and anchoring the ventricular anchor to the ventricle includes using the ventricular anchor to anchor the tether to the ventricle so as to anchor the ventricular end of the net to the ventricle.

For some applications, at least partially covering the atrial surface of the single native cardiac leaflet with the net includes covering, with the net, only a portion of the atrial surface of single native cardiac leaflet, the portion including an annular edge of the single native cardiac leaflet.

For some applications, at least partially covering the atrial surface of the single native cardiac leaflet with the net includes covering, with the net, (a) a portion of the atrial surface of the single native cardiac leaflet, the portion including an annular edge of the single native cardiac leaflet, and (b) a free ventricular edge of the single native cardiac leaflet.

For some applications, the net defines a plurality of openings and the net, when in a fully-expanded planar configuration, has an area, including the plurality of openings, of at least 100 mm2.

For some applications, the net defines a plurality of openings having an average opening area of at least 4 mm2, when the net is in a fully-expanded planar configuration.

For some applications, the average opening area is at least 10 mm2.

For some applications, the net defines a plurality of openings, and two or more of the openings have respective opening areas different from one another, when the net is in a fully-expanded planar configuration.

For some applications, three or more of the openings have respective opening areas different from one another, when the net is in the fully-expanded planar configuration.

For some applications, five or more of the openings have respective opening areas different from one another, when the net is in the fully-expanded planar configuration.

For some applications, the opening area of a first one of the two or more of the openings equals at least 110% of the opening area of a second one of the two or more of the openings, when the net is in the fully-expanded planar configuration.

For some applications, the opening area of the first opening equals at least 120% of the opening area of the second opening, when the net is in the fully-expanded planar configuration.

For some applications, the opening areas of the plurality of openings vary along the net between the annular side and the ventricular end of the net.

For some applications, the opening areas of the plurality of openings decrease along the net between the annular side and the ventricular end of the net.

For some applications, the opening areas of the plurality of openings increase along the net between the annular side and the ventricular end of the net.

For some applications, an average of the opening areas of the plurality of openings on an annular half of the net is different from an average of the opening areas of the plurality of openings on a ventricular half of the net.

For some applications, the average of the opening areas of the plurality of openings on the annular half of the net is greater than the average of the opening areas of the plurality of openings on the ventricular half of the net.

For some applications, the average of the opening areas of the plurality of openings on the annular half of the net is less than the average of the opening areas of the plurality of openings on the ventricular half of the net.

For some applications:
the net includes a plurality of flexible elongate members arranged so as to define a plurality of openings, and
the net, when in a fully-expanded planar configuration, has a material surface area ratio of 5%-80%, the material surface area ratio defined as the ratio of material of the flexible elongate members to an area of the net, including the plurality of openings and the material of the flexible elongate members.

For some applications:
the net includes a plurality of flexible elongate members arranged so as to define a plurality of openings, and
at least partially covering the atrial surface of the single native cardiac leaflet with the net includes at least partially covering the atrial surface of the single native cardiac leaflet with the net such that a net surface area ratio is 5%-150%, the net surface area ratio being the ratio of an area of the net, including the openings and material of the net, to a total area of the atrial surface.

For some applications, the net, when in a fully-expanded planar configuration, includes a triangular portion.

For some applications, the net, when in a fully-expanded planar configuration, is triangular.

For some applications, the net, when in a fully-expanded planar configuration, includes a rectangular portion.

For some applications, the net, when in a fully-expanded planar configuration, is rectangular.

For some applications, the net includes a plurality of flexible elongate members arranged at least partially as a grid when in a fully-expanded planar configuration.

For some applications, the net includes a plurality of flexible elongate members arranged at least partially in a spider-web configuration when in a fully-expanded planar configuration.

For some applications:
the leaflet restraint is a first leaflet restraint, the non-blood-occlusive net is a first non-blood-occlusive net, the two or more tissue-penetrating annulus anchors are two or more first tissue-penetrating annulus anchors, and the ventricular anchor is a first ventricular anchor, and
the method further includes:
   anchoring two or more second tissue-penetrating annulus anchors of a second leaflet restraint to the annulus of the native atrioventricular valve along or within 1 cm of a portion of the annulus to which the single native cardiac leaflet attaches;
   at least partially covering the atrial surface of the single native cardiac leaflet with a second non-blood-occlusive net of the second native leaflet restraint, the second tissue-penetrating annular anchors are fixed to an annular side of the second net; and
   anchoring a second ventricular anchor of the second leaflet restraint to the ventricle so as to anchor a ventricular end of the second net to the ventricle, such that the first and the second leaflet restraints limit leaflet with prolapse of the single native cardiac leaflet into the atrium.

The present invention will be more fully understood from the following detailed description of embodiments thereof, taken together with the drawings, in which:

### BRIEF DESCRIPTION OF THE DRAWINGS

FIGS. 1A-Q are schematic illustrations of several configurations of a leaflet restraint, in accordance with respective applications of the present invention;

FIGS. 2A-B are schematic illustrations of the leaflet restraint of FIGS. 1A-Q anchored within a heart, during systole and diastole, respectively, in accordance with an application of the present invention;

FIGS. 3A-B are schematic illustrations of configurations of a ventricular anchor, in accordance with respective applications of the present invention;

FIG. 4A-E are highly schematic illustrations of respective exemplary deployments of the native leaflet restraint of FIGS. 1A-Q, in accordance with an application of the present invention;

FIGS. 5A-B are schematic illustrations of the leaflet restraint of FIGS. 1A-Q anchored to a posterior native leaflet, in accordance with an application of the present invention;

FIGS. 6A-C are schematic illustrations of a method for treating a single native cardiac leaflet of a native atrioventricular valve, in accordance with an application of the present invention;

FIGS. 7A-C are schematic illustrations of another method for treating a single native cardiac leaflet of a native atrioventricular valve, in accordance with an application of the present invention;

FIGS. 8A-C are schematic cross-sectional illustrations of a cutting tool, in accordance with an application of the present invention; and

FIGS. 9A-C are schematic cross-sectional illustrations of another configuration of the cutting tool of FIGS. 8A-C, in accordance with an application of the present invention.

### DETAILED DESCRIPTION OF EMBODIMENTS

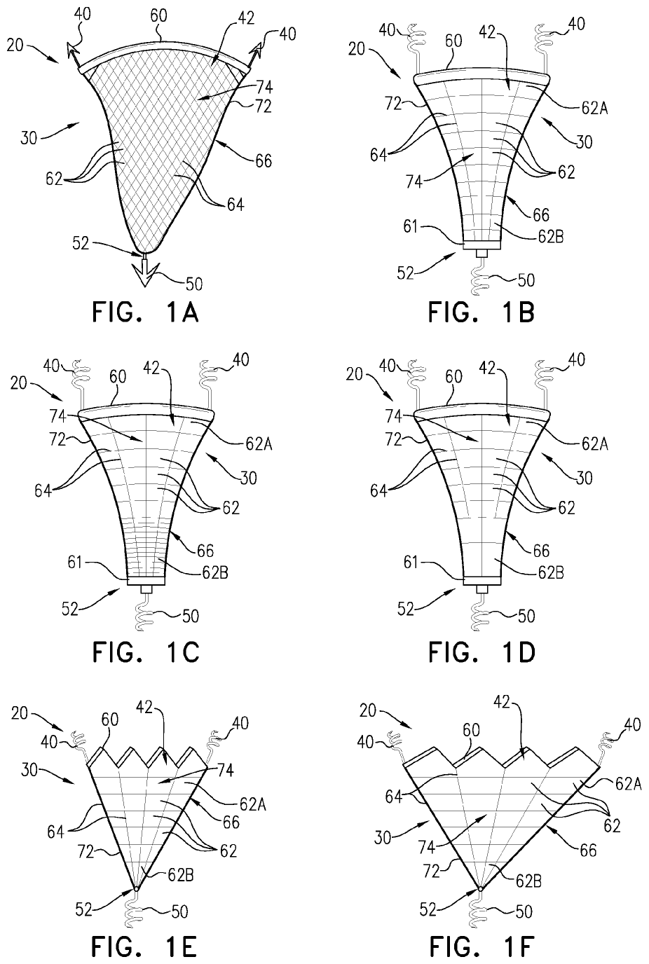

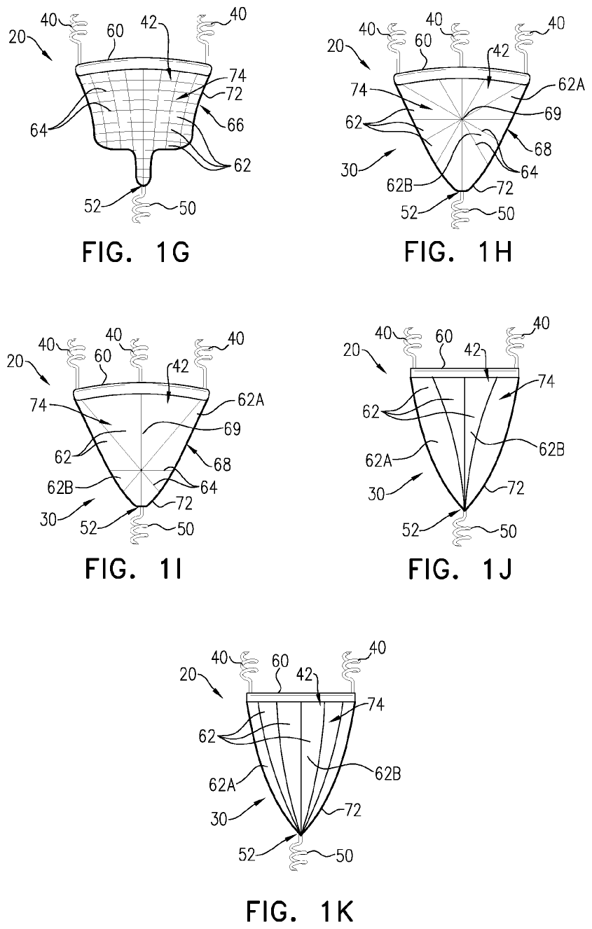

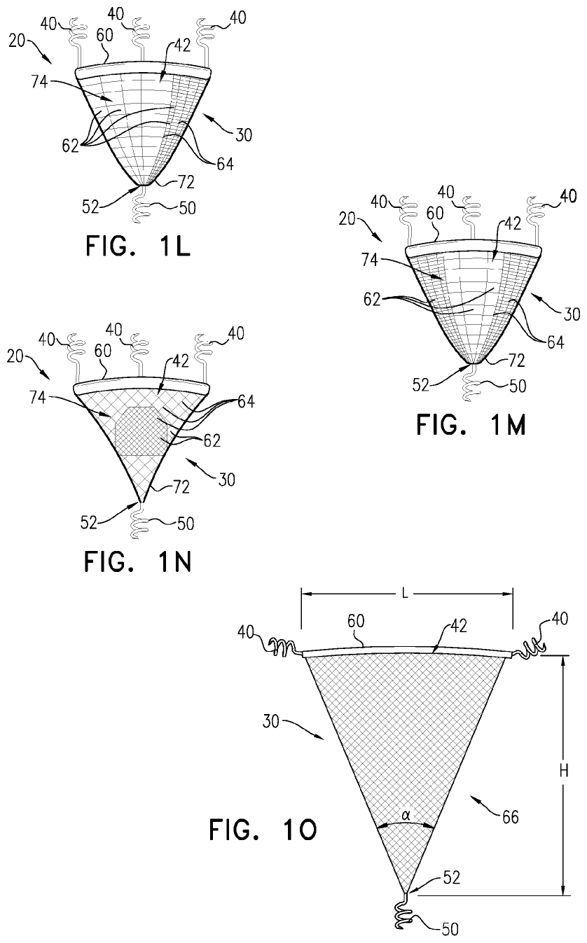

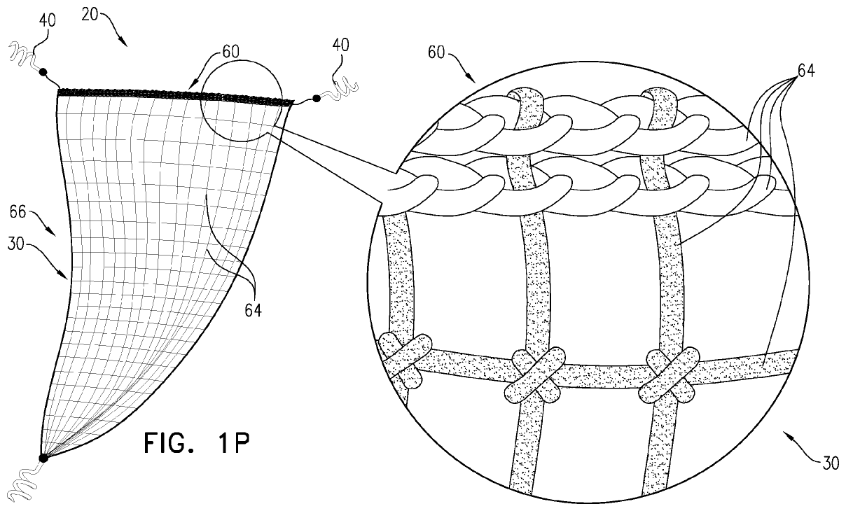

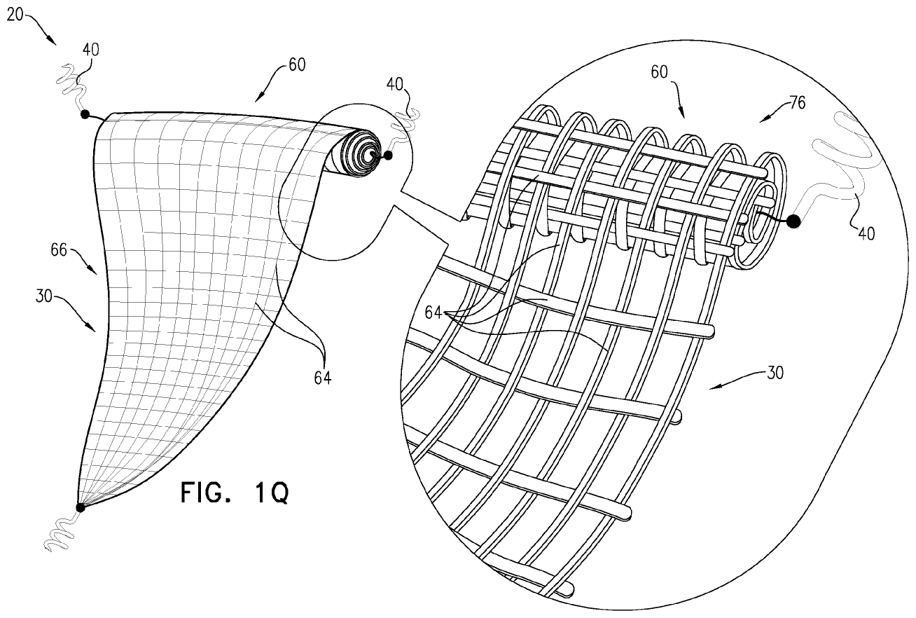

Reference is now made to FIGS. 1A-Q, which are schematic illustrations of several configurations of a leaflet restraint 20, in accordance with respective applications of the present invention.

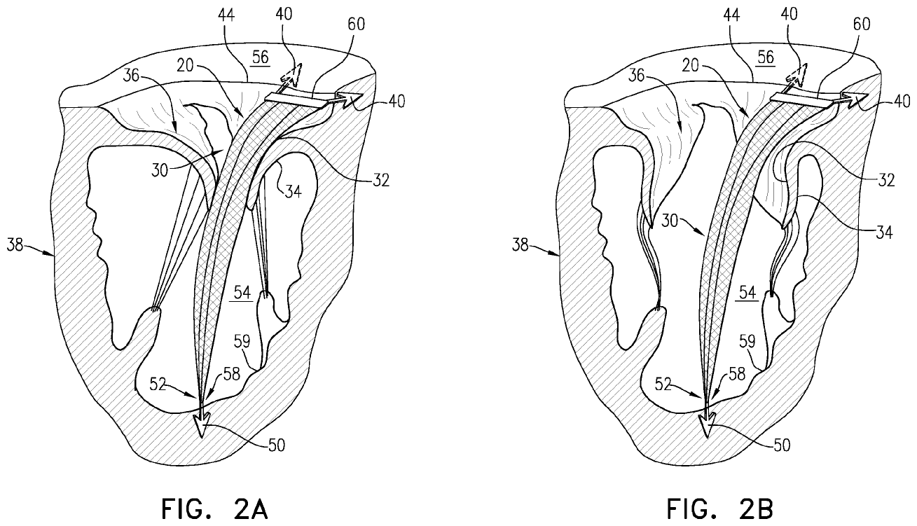

Reference is also made to FIGS. 2A-B, which are schematic illustrations of leaflet restraint 20 anchored within a heart 38, during systole and diastole, respectively, in accordance with an application of the present invention.

Leaflet restraint 20 comprises:
a non-blood-occlusive net 30, which is configured to at least partially cover an atrial surface 32 of a single native cardiac leaflet 34 of a single native atrioventricular valve 36 of heart 38 (at least during systole); for example, single native atrioventricular valve 36 may be a mitral valve, as shown, or a tricuspid valve (not shown);
two or more tissue-penetrating annulus anchors 40, which are fixed to an annular side 42 of net 30, and which are configured to be anchored to an annulus 44 of single native atrioventricular valve 36, typically along or within 1 cm of a portion of annulus 44 to which single native cardiac leaflet 34 attaches; and
a ventricular anchor 50, which is configured to be anchored to a ventricle 54 of heart 38 so as to anchor a ventricular end 52 of net 30 to ventricle 54.

Leaflet restraint 20 is configured, when anchored in place at least partially covering (and touching) atrial surface 32 of single native cardiac leaflet 34, to limit prolapse of single native cardiac leaflet 34 into an atrium 56 of heart 38.

Typically, leaflet restraint 20 does not comprise any annulus anchors that are configured to be anchored to annulus 44 other than along or within 1 cm of the portion of annulus 44 to which single native cardiac leaflet 34 attaches. Alternatively, leaflet restraint comprises one or more additional annulus anchors that are configured to be anchored to annulus 44 other than along or within 1 cm of the portion of annulus 44 to which single native cardiac leaflet 34 attaches.

It is noted that during diastole, such as shown in FIG. 2B, net 30 typically touches less of atrial surface 32 of single native cardiac leaflet 34 than during systole, such as shown in FIG. 2A; for example, net 30 may touch only a relatively small portion of atrial surface 32 near annulus 44 of single native atrioventricular valve 36.

As used in the present application, including in the claims, a “net” is an open-meshed fabric comprising flexible elongate members that are twisted, knotted, woven, knitted, braided, stitched, fused, or looped together at regular or irregular intervals. For example, flexible elongate members 64 may comprise wires, sutures, yarns, cords, threads, or ropes. For some applications, flexible elongate members 64 comprise wires or sutures. For example, the wires may comprise coatings of platinum, tantalum, or gold clad to interior materials such as copper, stainless steel, MP35N, Nitinol, nickel-iron, niobium, palladium, tantalum, titanium, or other alloys, or the sutures may comprise poliglecaprone 25 (e.g., Monocryl® (Ethicon)), nylon, polypropylene (e.g., PROLENE® (Ethicon)), silk, or polyglactin 910 (e.g., Vicryl® (Ethicon)). The suture material may be absorbable or non-absorbable, and/or monofilament or multifilament. In configurations in which flexible elongate members 64 cross one another in net 30 (e.g., as shown in FIGS. 1A-I and 1L-Q), flexible elongate members 64 may or may not be fixed to one another (e.g., knotted) at the crossing junctions; optionally, flexible elongate members 64 are fixed to one another at only a portion of the crossing junctions.

For some applications, flexible elongate members 64 have an average diameter of at least 100 microns, no more than 3000 microns, and/or 100-3000 microns.

For some applications, flexible elongate members 64 that define one or more borders 72 of net 30 are thicker and/or more rigid than flexible elongate members 64 that define an internal, non-border region 74 of net 30. The one or more borders 72 of net 30 may help define and maintain the shape of the net.

For example, flexible elongate members 64 that define the one or more borders 72 of net 30 may have an average diameter of:
25-100% (e.g., 25-50%) of an average diameter of flexible elongate members 64 that define internal, non-border region 74 of net 30; and/or
500-3000 microns.

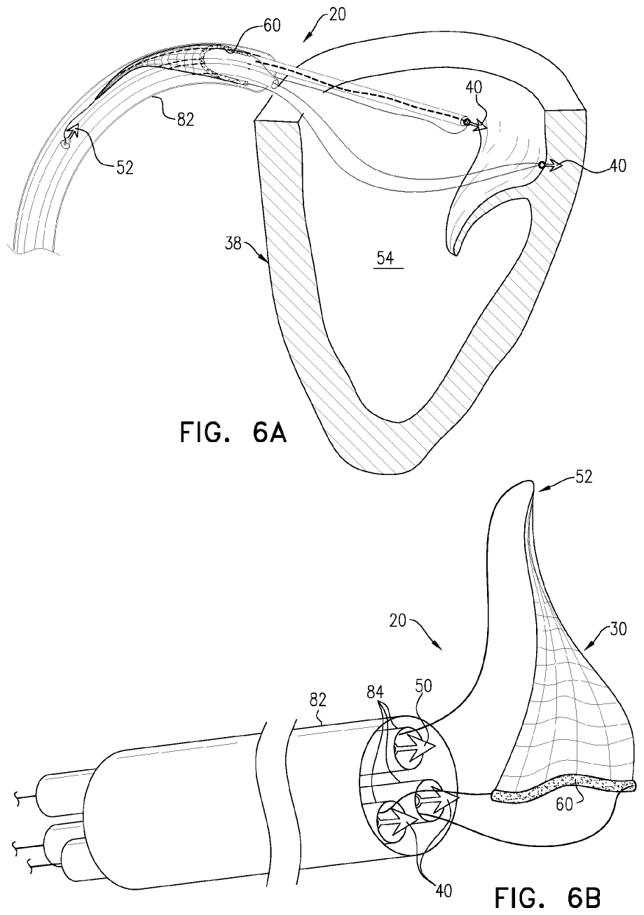

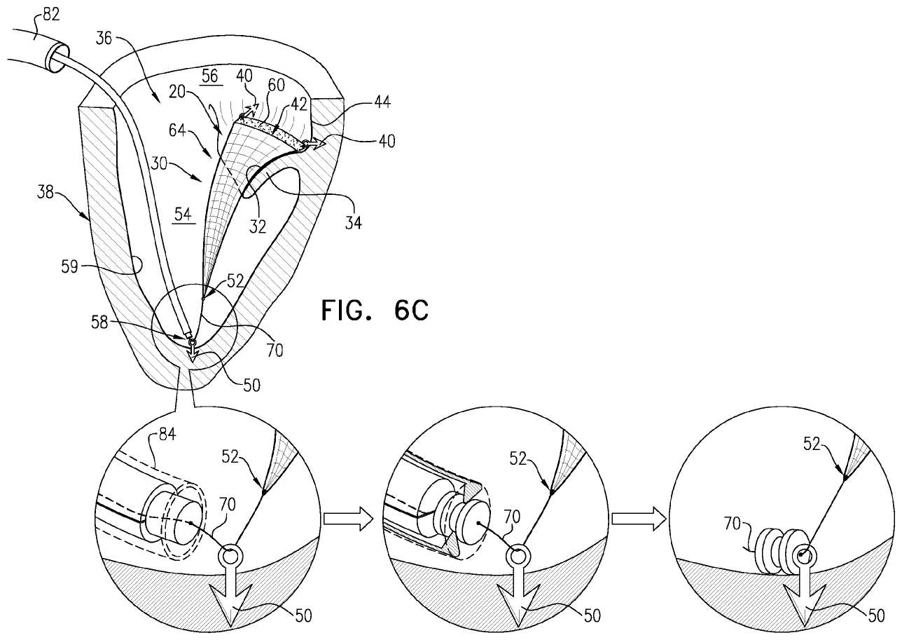

For some applications, ventricular anchor 50 is fixed to ventricular end 52 of net 30, either directly, such as shown in FIGS. 1A-Q, 2A-B, and 5A-B, or indirectly, such as via a tether. For other applications, ventricular anchor 50 is provided separately from net 30, and is coupled to ventricular end 52 of net 30 during an implantation procedure, such as described hereinbelow with reference to FIGS. 6A-C.

For some applications, ventricular anchor 50 is configured to be anchored to a ventricular site 58 within ventricle 54, the site selected from the group of sites consisting of: a papillary muscle (configuration not shown), a chorda tendinea (configuration not shown), a free edge of another native cardiac leaflet (configuration not shown), a ventricular wall 59 (as illustrated), and ventricular trabeculae (configuration not shown). (The free edge of another native cardiac leaflet is considered to be a ventricular site because the free edge is disposed below the level of the annulus.) For example, ventricular site 58 may be located on an apical third of ventricular wall 59, such as at or near the apex, as shown.

For some applications, leaflet restraint 20 further comprises an annular support 60 that extends along at least a portion of annular side 42 of net 30, in order to maintain a length of annular side 42 of net 30 and/or to maintain a shape of annular side 42 of net 30. Typically, the two or more tissue-penetrating annulus anchors 40 are fixed with respect to annular support 60; for example, the two or more tissue-penetrating annulus anchors 40 may be fixed directly to annular support 60.

For some applications, such as shown in FIGS. 1A-D and 1G-O, annular support 60 is defined by a discrete element, such as a rod, which is fixed to net 30.

For other applications, such as shown in FIGS. 1P-Q, annular support 60 is defined by the at least a portion of annular side 42 of net 30, and wherein flexible elongate members 64 of the at least a portion of annular side 42 of net 30 are arranged so to provide annular support 60 with greater rigidity than other portions of net 30. For example, flexible elongate members 64 of the at least a portion of annular side 42 of net 30 may be:
woven so to provide annular support 60 with greater rigidity than other portions of net 30, such as shown in FIG. 1P, or
rolled in a scroll-shaped arrangement 76 so to provide annular support 60 with the greater rigidity than other portions of net 30, such as shown in FIG. 1Q.

For some applications, leaflet restraint 20 further comprises a ventricular-end support 61, disposed at ventricular end 52 of net 30, which may help maintain a shape of ventricular end 52 and/or provide better coupling of ventricular anchor 50 to ventricular end 52 of net 30.

For some applications, net 30 defines a plurality of openings 62 having an average opening area of at least 4 mm2 (such as at least 10 mm2) and/or no more than 25 mm2, when net 30 is in a fully-expanded planar configuration, such as shown in FIGS. 1A-Q.

Typically, net 30 has an area (including openings 62 and the material of net 30) of at least 75 mm2 (such as at least 100 mm2) and/or no more than 200 mm2, when net 30 is in a fully-expanded planar configuration, such as shown in FIGS. 1A-Q.

In some applications of the present invention, a particular configuration of net 30 is selected or fabricated based on the particular anatomical issues of each patient that contribute to the prolapse of single native cardiac leaflet 34 into an atrium 56 of heart 38. By way of example and not limitation, the particular configurations of net 30 illustrated in FIGS. 1A-Q may be used to address these individual anatomical issues.

For some applications, when net 30 is in a fully-expanded planar configuration, net 30 comprises a triangular portion, such as shown in FIGS. 1A, 1E, 1F, 1H, 1I, 1J, and IK-Q. For some of these applications, net 30 is triangular, such as shown in FIGS. 1A, 1E, 1F, 1H, 1I, 1J, and 1K-Q.

For some applications, when net 30 is in a fully-expanded planar configuration, net 30 is generally triangular, e.g., trapezoidal with the legs substantially longer than the short base, such as shown in FIGS. 1B, 1C, and 1D.

For some applications, when net 30 is in a fully-expanded planar configuration, net 30 comprises a rectangular portion, such as shown in FIG. 1G. For some of these applications, net 30 is rectangular (configuration not shown).

For some applications, net 30 has a material surface area ratio of at least 5%, no more than 80% (e.g., no more than 25%), and/or 5%-80% (e.g., 5%-25%), when net 30 is in a fully-expanded planar configuration, such as shown in FIGS. 1A-Q. The material surface area ratio is the ratio of the area of the material of flexible elongate members 64 to the area of net 30 (including openings 62 and the material of net 30).

For some applications, when net 30 is in a fully-expanded planar configuration, the plurality of flexible elongate members 64 are arranged at least partially as a grid 66, such as shown in FIGS. 1A-G and 1O-Q.

For some applications, when net 30 is in a fully-expanded planar configuration, the plurality of flexible elongate members 64 are arranged at least partially in a spider-web configuration 68, such as shown in FIGS. 1H and 1I, in which the plurality of flexible elongate members 64 are typically fixed together (e.g., knotted) at only a single junction 69 in a central area of spider-web configuration 68, which may provide greater central restraint of single native cardiac leaflet 34 than peripheral restraint. Typically, flexible elongate members 64 are also arranged to provide an external border 72 to support spider-web configuration 68.

For some applications, when net 30 is in a fully-expanded planar configuration, the plurality of flexible elongate members 64 are arranged at least partially extending from annular side 42 of net 30 to ventricular end 52 of net 30, such as shown in FIGS. 1J and 1K. For example, the plurality of flexible elongate members 64 may be arranged similar to a Witch's broom (parachute) string game, such as shown in FIGS. 1J and 1K. The atrial ends of flexible elongate members 64 may be evenly distributed along annular support 60, such as shown in FIG. 1J, or unevenly distributed along annular support 60, such as shown in FIG. 1K.

Reference is made to FIGS. 1B-F and 1H-N. For some applications, two or more of the openings 62 (e.g., three or more, five or more, or ten or more) have respective opening areas different from one another, when net 30 is in the fully-expanded planar configuration.

For some of these applications, such as labeled by way of example in FIGS. 1B-F and 1H-K, the opening area of a first one 62A of openings 62 equals at least 110% (i.e., is at least 10% greater than) (e.g., at least 120%, such as at least 150%) of the opening area of a second one 62B of openings 62, when net 30 is in the fully-expanded planar configuration.

For some applications, the opening areas of the plurality of openings 62 vary along net 30 between annular side 42 and ventricular end 52 of net 30, such as shown, for example, in FIGS. 1B-F and 1H-I. For example:
the opening areas of the plurality of openings 62 may decrease along net 30 between annular side 42 and ventricular end 52 of net 30, such as shown in FIGS. 1B, 1C, 1E, 1F, and 1I,
the average of the opening areas of the plurality of openings 62 on an annular half of net 30 may be greater than the average of the opening areas of the plurality of openings 62 on a ventricular half of net 30, such as shown in FIGS. 1B, 1C, 1E, 1F, and 1I,
the opening areas of the plurality of openings 62 may increase along net 30 between annular side 42 and ventricular end 52 of net 30, such as shown in FIG. 1H,
the average of the opening areas of the plurality of openings 62 on an annular half of net 30 may be less than the average of the opening areas of the plurality of openings 62 on a ventricular half of net 30, such as shown in FIGS. 1D and 1H, or
the average widths of the plurality of openings 62, measured perpendicular to an atrial-ventricular direction, may decrease along net 30 between annular side 42 and ventricular end 52 of net 30, such as shown in FIGS. 1J and 1K.

For other applications, the opening areas of some (e.g., at least 50% of, such as 100%) of the plurality of openings 62 equal one another, or approximately equal another (e.g., vary by less than 10%), such as shown, for example, in FIGS. 1A, 1G, and 1O-Q.

For some applications, a density of net 30 may be greater in a specific target region, which may, for example, be lateral to the main net region (as in FIG. 1L) or in a coaptation region (as in FIG. 1N).

Reference is now made to FIG. 1O. In this configuration, net 30 is triangular when in the fully-expanded planar configuration. For some applications, the triangle of net 30 has one or more of the following dimensions:
a height H of at least 45 mm, no more than 70 mm, and/or 45-70 mm, such as 55 mm;
a base length L of at least 35 mm, no more than 55 mm, and/or 35-55 mm, such as 40 mm; and/or
an angle α (alpha) at ventricular end 52 of net 30 of at least 25 degrees, no more than 40 degrees, and/or 25-40 degrees, e.g., 30 degrees.

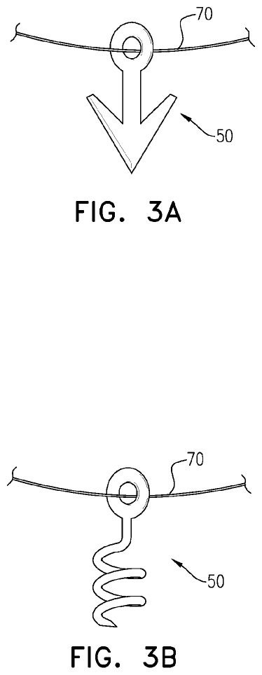

Reference is now made to FIGS. 3A-B, which are schematic illustrations of configurations of ventricular anchor 50, in accordance with respective applications of the present invention.

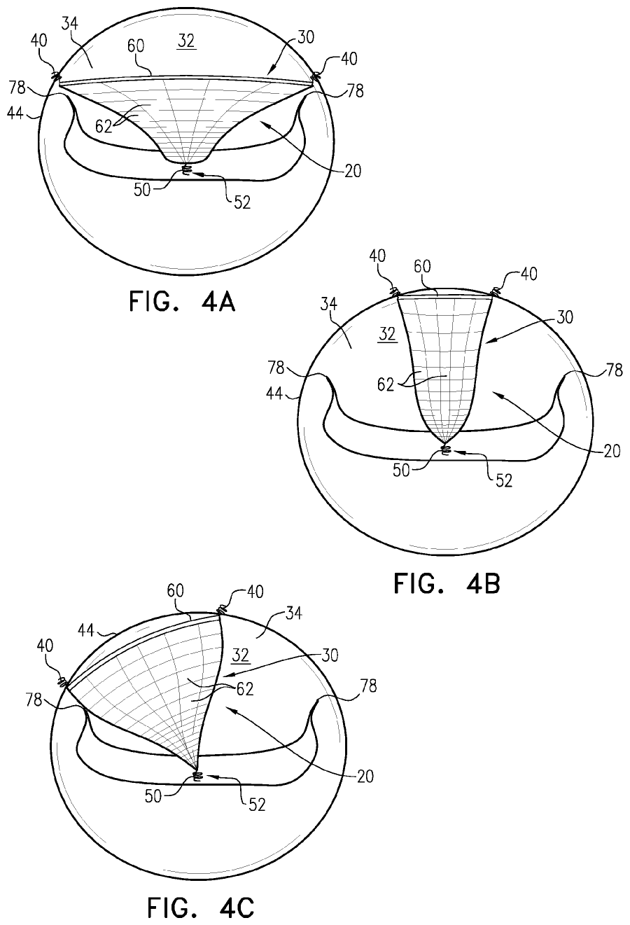

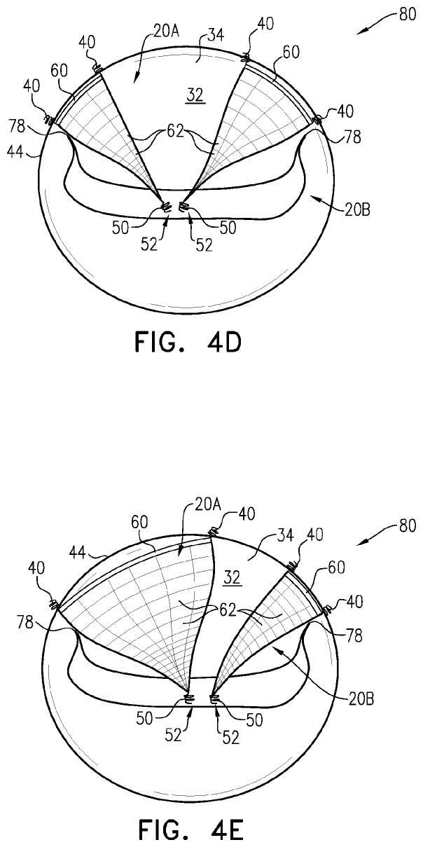

Reference is now made to FIG. 4A-E, which are highly schematic illustrations of respective exemplary deployments of native leaflet restraint 20, in accordance with an application of the present invention. In some applications of the present invention, the placement of native leaflet restraint 20 is targeted based on the particular anatomical issues of each patient. For example, one or more portions of one or more native leaflet restraints 20 may be placed to restrain one or more particular respective portions of single native atrioventricular valve 36 that contribute to the prolapse of single native cardiac leaflet 34 into an atrium 56 of heart 38. By way of example and not limitation, the particular configurations of net 30 illustrated in FIGS. 4A-E may be used to address these individual anatomical issues. Optionally, this targeted placement is implemented in combination with the selection of a particular configuration of net 30 based on the particular anatomical issues of the patient, as described hereinabove with reference to FIGS. 1A-Q.

For some applications, net 30 is anchored by tissue-penetrating annulus anchors 40 commissure-to-commissure of single native atrioventricular valve 36, such as shown FIG. 4A.

For some applications, net 30 is anchored by tissue-penetrating annulus anchors 40 to annulus 44 of single native atrioventricular valve 36 along a central portion of annulus 44 between commissures 78, such as shown FIG. 4B.

For some applications, net 30 is anchored by tissue-penetrating annulus anchors 40 to annulus 44 of single native atrioventricular valve 36 at one commissure 78 and at another location between commissures 78, such as shown FIG. 4C.

For some applications, such as shown in FIG. 4B, net 30 is configured to cover only a portion of atrial surface 32 of single native cardiac leaflet 34, the portion including an annular edge of single native cardiac leaflet 34.

For other applications, such as shown in FIGS. 4C, net 30 is configured to cover (a) a portion of atrial surface 32 of single native cardiac leaflet 34, the portion including an annular edge of single native cardiac leaflet 34, and (b) a free ventricular edge of single native cardiac leaflet 34.

For some applications, such as shown in FIGS. 4D and 4E, a leaflet-restraint system 80 is provided that comprises leaflet restraint 20 as a first leaflet restraint 20A, and further comprises a second leaflet restraint 20B. Second leaflet restraint 20B may be identical to first leaflet restraint 20A (configuration not shown), or may have different a different configuration (e.g., a different size and/or shape), such as shown in FIGS. 4D-E.

Typically, first and second leaflet restraints 20A and 20B are configured to be anchored in place partially covering atrial surface 32 of the same single native cardiac leaflet 34, so as to limit prolapse of single native cardiac leaflet 34 into atrium 56. Alternatively, first and second leaflet restraints 20A and 20B are configured to be anchored in place partially covering respective atrial surfaces of two native cardiac leaflets 34 (configuration not shown).

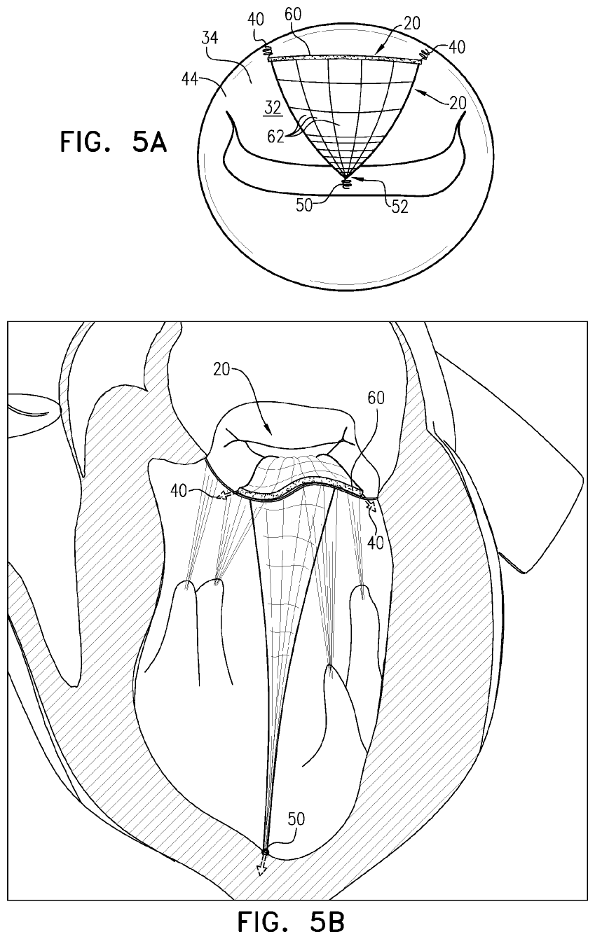

Reference is now made to FIGS. 5A-B, which are schematic illustrations of leaflet restraint 20 anchored to posterior native cardiac leaflet 34, in accordance with an application of the present invention. Leaflet restraint 20 further comprises annular support 60, which is pre-shaped to adapt to the annular anatomy. For example, annular support 60 may be shaped so as to define one or more curves that define peaks and/or troughs that are oriented in a superior and/or inferior direction, respectively, and are shaped to match the superior-inferior contour of the native annular anatomy.

Reference is now made to FIGS. 6A-C, which are schematic illustrations of a method for treating single native cardiac leaflet 34 of single native atrioventricular valve 36 of heart 38, in accordance with an application of the present invention.

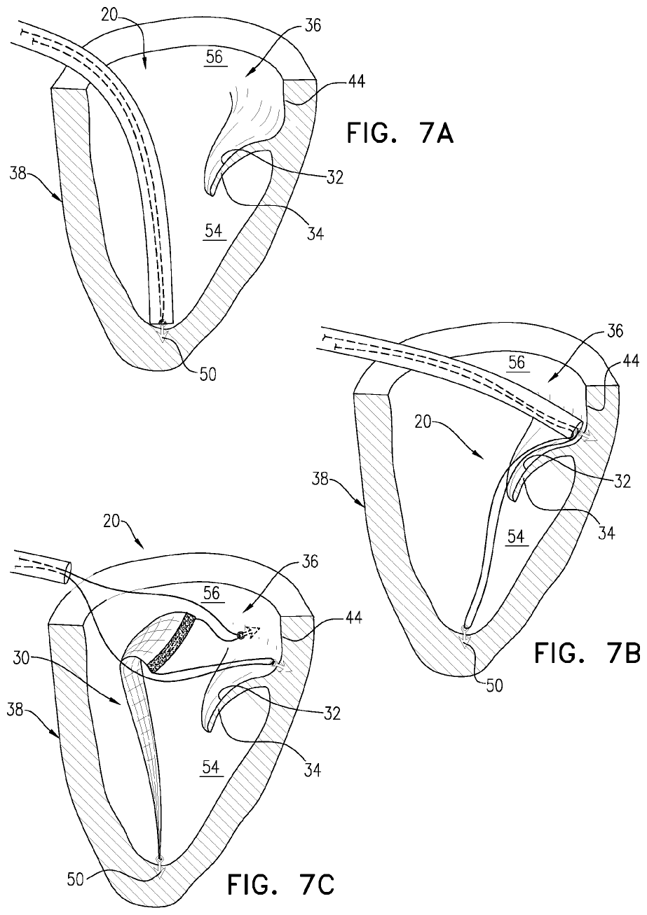

Reference is also made to FIGS. 7A-C, which are schematic illustrations of another method for treating single native cardiac leaflet 34 of single native atrioventricular valve 36 of heart 38, in accordance with an application of the present invention.

In both approaches, the method comprises:
anchoring two or more tissue-penetrating annulus anchors 40 to annulus 44 of single native atrioventricular valve 36, such as shown in FIG. 6A;
at least partially covering atrial surface 32 of single native cardiac leaflet 34 with non-blood-occlusive net 30 of native leaflet restraint 20, such as shown in FIG. 6C; and
anchoring ventricular anchor 50 to a ventricle of heart 38, such that leaflet restraint 20 limits prolapse of single native cardiac leaflet 34 into atrium 56, such as shown in FIG. 6C.

These steps may be performed in any order.

For some applications, atrial surface 32 is at least partially covered with net 30 such that a net surface area ratio is at least 5% (e.g., at least 50%), no more than 150% (e.g., no more than 120%, or no more than 100%), and/or 5-150%, such as 50-150%. The net surface area ratio is the ratio of the area of net 30 (including openings 62 and the material of net 30) to the total area of atrial surface 32.

For some applications, ventricular anchor 50 is anchored at a ventricular site 58 within ventricle 54 that sets a desired angle of a best-fit plane defined by net 30 with respect to a plane of the annulus, in order to set a level of restraint of single native cardiac leaflet 34. As used in the present application, including in the claims, a “best-fit plane” of net 30 is the plane that results in the minimum sum of squares of distances between the plane and all points of net 30.

The method is typically performed using a transvascular, transcatheter approach, such as shown in FIGS. 6A-C. A catheter 82 is advanced into atrium 56, using techniques known in the art, and leaflet restraint 20 is deployed from the catheter. For some applications, separate inner catheters 84 are provided for deploying each of anchors 40 and 50, such as shown in FIGS. 6A-C, perhaps most clearly in FIG. 6B.

For some applications, such as shown in FIG. 6B, leaflet restraint 20 further comprises a tether 70 that is fixed to ventricular end 52 of net 30, and ventricular anchor 50 is configured to anchor tether 70 to ventricle 54 so as to anchor ventricular end 52 of net 30 to ventricle 54. For some of these applications, upon completion of the implantation procedure, a portion of tether 70 remains between ventricular anchor 50 and ventricular end 52 of net 30, such as shown in FIG. 6C, while for other applications, tether 70 is pulled through the head of ventricular anchor 50 until ventricular end 52 of net 30 reaches the ventricular anchor, such as shown in FIG. 5B, or is even pulled through the head of ventricular anchor 50 (configuration not shown). For some applications, pulling ventricular end 52 of net 30 through the head of ventricular anchor 50 locks net 30 to ventricular anchor.

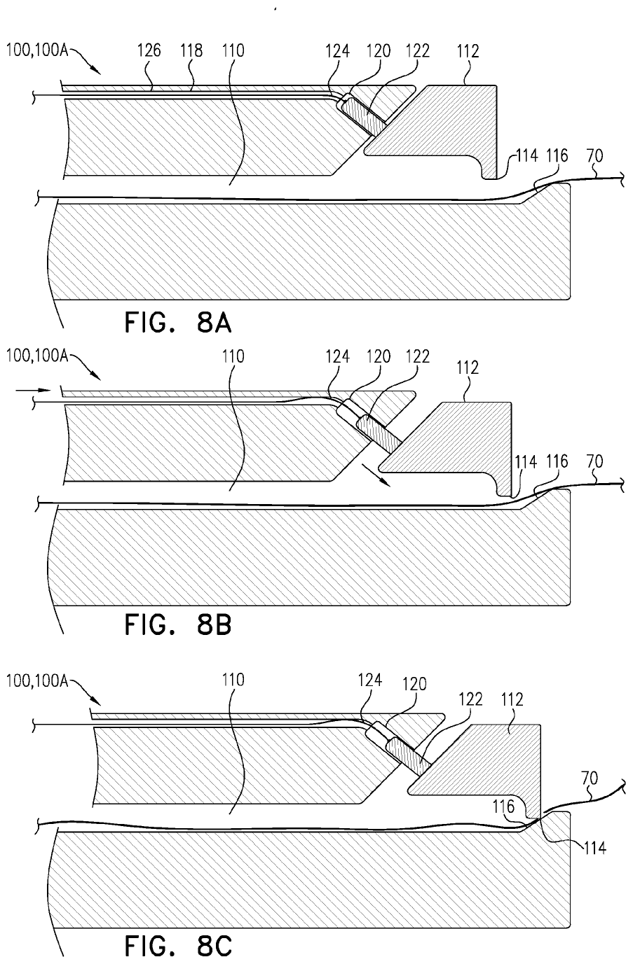

For some applications, tether 70 is pulled through the head of ventricular anchor 50 until a desired degree of anterior pulling on net 30 is achieved, and tether 70 is locked with respect to the head of ventricular anchor 50 by crimping. For example, a cylindrical crimp 86 may be passed over tether 70 via one of the catheters, and then crimped using a delivery tool, such as shown in FIG. 6C. Excess tether may be cut using suture-cutting techniques known in the art, or using the cutting techniques described hereinbelow with reference to FIGS. 8A-C or 9A-C, which may optionally be integrated into a distal portion of inner catheter 84 of the delivery tool, as shown.

Reference is now made to FIGS. 8A-C or 9A-C, which are schematic cross-sectional illustrations of a cutting tool 100, in accordance with respective applications of the present invention. FIGS. 8A-C or 9A-C show a distal portion of cutting tool 100. Cutting tool 100 is configured to be advanced distally (to the right in the drawings) over tether 70, while tether 70 passes through a channel 110 defined by cutting tool 100. Cutting tool 100 comprises a cutter 112, which is shaped so as to define a sharp cutting surface 114. Cutting tool 100 is configured to advance cutter 112 distally and radially inwardly so as to trap tether 70 between cutter 112 and an opposing surface 116 of cutting tool 100, thereby severing tether 70.

For some applications, cutting tool 100 is shaped so as to define a control channel 118, which includes a distal portion 120 that is angled radially inwardly in the same direction as the distal and radially inward motion of cutter 112. A shaft 122 supports cutter 112 and is disposed within distal portion 120 of control channel 118. Cutting tool 100 comprises a control wire 124 that passes through a proximal portion 126 of control channel 118 and is coupled to shaft 122.

For some applications, such as shown for cutting tool 100A in FIGS. 8A-C, control wire 124 is sufficiently rigid such that distal advancement of control wire 124 pushes a shaft 122, 122A within distal portion 120 of control channel 118.

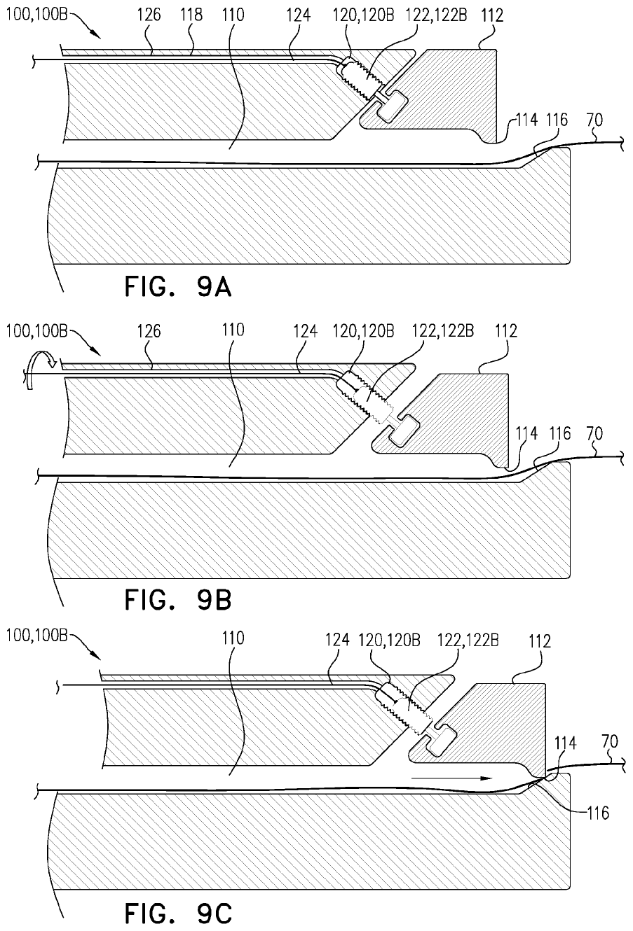

For other applications, such as shown for cutting tool 100B in FIGS. 9A-C, control wire 124 is sufficiently rigid such that rotation of control wire 124 rotates a threaded shaft 122, 122B within distal portion 120, 120B of control channel 118. Distal portion 120, 120B of control channel 118 is threaded, such that the rotation of threaded shaft 122, 112B causes the shaft to advance distally within distal portion 120, 120B of the control channel.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A leaflet restraint comprising:
a non-blood-occlusive net, which is configured to at least partially cover an atrial surface of a single native cardiac leaflet of a native atrioventricular valve of a heart;
two or more tissue-penetrating annulus anchors, which are fixed to an annular side of the net, and which are configured to be anchored to an annulus of the native atrioventricular valve along or within 1 cm of a portion of the annulus to which the single native cardiac leaflet attaches; and
a ventricular anchor, which is configured to be anchored to a ventricle of the heart so as to anchor a ventricular end of the net to the ventricle,
wherein the leaflet restraint does not comprise any annulus anchors that are configured to be anchored to the annulus other than along or within 1 cm of the portion of the annulus to which the single native cardiac leaflet attaches, and
wherein the leaflet restraint is configured, when anchored in place at least partially covering the atrial surface of the single native cardiac leaflet, to limit prolapse of the single native cardiac leaflet into an atrium of the heart.

2. The leaflet restraint according to claim 1, wherein the ventricular anchor is configured to be anchored to a ventricular site within the ventricle, the site selected from the group of sites consisting of: a papillary muscle, a chorda tendinea, a free edge of another native cardiac leaflet, a ventricular wall, and ventricular trabeculae.

3. The leaflet restraint according to claim 1, wherein the ventricular anchor is configured to be anchored to the ventricle so as to directly anchor the ventricular end of the net to the ventricle.

4. The leaflet restraint according to claim 1, further comprising a tether that is fixed to the ventricular end of the net, wherein the ventricular anchor is configured to anchor the tether to the ventricle so as to anchor the ventricular end of the net to the ventricle.

5. The leaflet restraint according to claim 1, wherein the net is configured to cover only a portion of the atrial surface of the single native cardiac leaflet, the portion including an annular edge of the single native cardiac leaflet.

6. The leaflet restraint according to claim 1, wherein the net is configured to cover (a) a portion of the atrial surface of the single native cardiac leaflet, the portion including an annular edge of the single native cardiac leaflet, and (b) a free ventricular edge of the single native cardiac leaflet.

7. The leaflet restraint according to claim 1, wherein the net defines a plurality of openings and the net, when in a fully-expanded planar configuration, has an area, including the plurality of openings, of at least 100 mm2.

8. The leaflet restraint according to claim 1,
wherein the net comprises a plurality of flexible elongate members arranged so as to define a plurality of openings, and
wherein the net, when in a fully-expanded planar configuration, has a material surface area ratio of 5%-80%, the material surface area ratio being the ratio of the area of material of the flexible elongate members to an area of the net, including the plurality of openings and the material of the flexible elongate members.

9. The leaflet restraint according to claim 1, wherein the net, when in a fully-expanded planar configuration, comprises a triangular portion.

10. The leaflet restraint according to claim 1, wherein the net, when in a fully-expanded planar configuration, is triangular.

11. The leaflet restraint according to claim 1, wherein the net, when in a fully-expanded planar configuration, comprises a rectangular portion.

12. The leaflet restraint according to claim 1, wherein the net, when in a fully-expanded planar configuration, is rectangular.

13. The leaflet restraint according to claim 1, wherein the net comprises a plurality of flexible elongate members arranged at least partially as a grid when in a fully-expanded planar configuration.

14. The leaflet restraint according to claim 1, wherein the net comprises a plurality of flexible elongate members arranged at least partially in a spider-web configuration when in a fully-expanded planar configuration.

15. The leaflet restraint according to any one of claims 1-14, further comprising an annular support that extends along at least a portion of the annular side of the net, in order to maintain a length of the annular side of the net.

16. The leaflet restraint according to claim 15, wherein the annular support is defined by a discrete element fixed to the at least a portion of the net.

17. The leaflet restraint according to claim 16, wherein the discrete element comprises a rod.

18. The leaflet restraint according to claim 15,
wherein the net comprises a plurality of flexible elongate members,
wherein the annular support is defined by the at least a portion of the annular side of the net, and
wherein the flexible elongate members of the at least a portion of the annular side of the net are arranged so to provide the annular support with greater rigidity than other portions of the net.

19. The leaflet restraint according to claim 18, wherein the flexible elongate members of the at least a portion of the annular side of the net are woven so to provide the annular support with greater rigidity than other portions of the net.

20. The leaflet restraint according to claim 18, wherein the flexible elongate members of the at least a portion of the annular side of the net are rolled in a scroll-shaped arrangement so to provide the annular support with the greater rigidity than other portions of the net.

21. The leaflet restraint according to claim 15, wherein the two or more tissue-penetrating annulus anchors are fixed with respect to the annular support.

22. The leaflet restraint according to claim 21, wherein the two or more tissue-penetrating annulus anchors are fixed directly to the annular support.

23. The leaflet restraint according to any one of claims 1-14, wherein the net defines a plurality of openings having an average opening area of at least 4 mm2, when the net is in a fully-expanded planar configuration.

24. The leaflet restraint according to claim 23, wherein the average opening area is at least 10 mm2.

25. The leaflet restraint according to any one of claims 1-14, wherein the net defines a plurality of openings, and wherein two or more of the openings have respective opening areas different from one another, when the net is in a fully-expanded planar configuration.

26. The leaflet restraint according to claim 25, wherein three or more of the openings have respective opening areas different from one another, when the net is in the fully-expanded planar configuration.

27. The leaflet restraint according to claim 26, wherein five or more of the openings have respective opening areas different from one another, when the net is in the fully-expanded planar configuration.

28. The leaflet restraint according to claim 25, wherein the opening area of a first one of the two or more of the openings equals at least 110% of the opening area of a second one of the two or more of the openings, when the net is in the fully-expanded planar configuration.

29. The leaflet restraint according to claim 28, wherein the opening area of the first opening equals at least 120% of the opening area of the second opening, when the net is in the fully-expanded planar configuration.

30. The leaflet restraint according to claim 25, wherein the opening areas of the plurality of openings vary along the net between the annular side and the ventricular end of the net.

31. The leaflet restraint according to claim 30, wherein the opening areas of the plurality of openings decrease along the net between the annular side and the ventricular end of the net.

32. The leaflet restraint according to claim 30, wherein the opening areas of the plurality of openings increase along the net between the annular side and the ventricular end of the net.

33. The leaflet restraint according to claim 25, wherein an average of the opening areas of the plurality of openings on an annular half of the net is different from an average of the opening areas of the plurality of openings on a ventricular half of the net.

34. The leaflet restraint according to claim 33, wherein the average of the opening areas of the plurality of openings on the annular half of the net is greater than the average of the opening areas of the plurality of openings on the ventricular half of the net.

35. The leaflet restraint according to claim 33, wherein the average of the opening areas of the plurality of openings on the annular half of the net is less than the average of the opening areas of the plurality of openings on the ventricular half of the net.

36. A leaflet-restraint system comprising the leaflet restraint according to any one of claims 1-14,
wherein the leaflet restraint is a first leaflet restraint, the non-blood-occlusive net is a first non-blood-occlusive net, the two or more tissue-penetrating annulus anchors are two or more first tissue-penetrating annulus anchors, and the ventricular anchor is a first ventricular anchor,
wherein the leaflet-restraint system further comprises a second leaflet restraint, which comprises:
a second non-blood-occlusive net, which is configured to at least partially cover the atrial surface of the single native cardiac leaflet;
two or more second tissue-penetrating annulus anchors, which are fixed to an annular side of the second net, and which are configured to be anchored to the annulus of the native atrioventricular valve along or within 1 cm of a portion of the annulus to which the single native cardiac leaflet attaches; and
a second ventricular anchor, which is configured to be anchored to the ventricle so as to anchor a ventricular end of the second net to the ventricle, and
wherein the first and the second leaflet restraints are configured, when anchored in place at least partially covering the atrial surface of the single native cardiac leaflet, to limit prolapse of the single native cardiac leaflet into the atrium.

37. A method for treating a single native cardiac leaflet of a native atrioventricular valve of a heart, the method comprising:
anchoring two or more tissue-penetrating annulus anchors of a leaflet restraint to an annulus of the native atrioventricular valve along or within 1 cm of a portion of the annulus to which the single native cardiac leaflet attaches;
at least partially covering an atrial surface of the single native cardiac leaflet with a non-blood-occlusive net of the native leaflet restraint, wherein the tissue-penetrating annulus anchors are fixed to an annular side of the net; and
anchoring a ventricular anchor of the leaflet restraint to a ventricle of the heart so as to anchor a ventricular end of the net to the ventricle, such that the leaflet restraint limits prolapse of the single native cardiac leaflet into an atrium of the heart,
wherein the method does not comprise anchoring any annulus anchors to the annulus other than along or within 1 cm of the portion of the annulus to which the single native cardiac leaflet attaches.

38. The method according to claim 37, wherein anchoring the ventricular anchor comprises anchoring the ventricular anchor at an anchoring site within the ventricle that sets a desired angle of a best-fit plane defined by the net with respect to a plane of the annulus, in order to set a level of restraint of the single native cardiac leaflet.

39. The method according to claim 37, wherein anchoring the ventricular anchor to the ventricle comprises anchoring the ventricular anchor to a ventricular site within the ventricle, the site selected from the group of sites consisting of: a papillary muscle, a chorda tendinea, a free edge of another single native cardiac leaflet, a ventricular wall, and ventricular trabeculae.

40. The method according to claim 37, wherein the native leaflet restraint further includes an annular support that extends along at least a portion of the annular side of the net, in order to maintain a length of the annular side of the net.

41. The method according to claim 40, wherein the annular support is defined by a discrete element fixed to the at least a portion of the net.

42. The method according to claim 41, wherein the discrete element comprises a rod.

43. The method according to claim 40,
wherein the net comprises a plurality of flexible elongate members,
wherein the annular support is defined by the at least a portion of the annular side of the net, and
wherein the flexible elongate members of the at least a portion of the annular side of the net are arranged so to provide the annular support with greater rigidity than other portions of the net.

44. The method according to claim 43, wherein the flexible elongate members of the at least a portion of the annular side of the net are woven so to provide the annular support with greater rigidity than other portions of the net.

45. The method according to claim 43, wherein the flexible elongate members of the at least a portion of the annular side of the net are rolled in a scroll-shaped arrangement so to provide the annular support with the greater rigidity than other portions of the net.

46. The method according to claim 40, wherein the two or more tissue-penetrating annulus anchors are fixed with respect to the annular support.

47. The method according to claim 46, wherein the two or more tissue-penetrating annulus anchors are fixed directly to the annular support.

48. The method according to claim 37, wherein anchoring the ventricular anchor to the ventricle comprising using the ventricular anchor to directly anchor the ventricular end of the net to the ventricle.

49. The method according to claim 37, wherein the leaflet restraint further includes a tether that is fixed to the ventricular end of the net, and wherein anchoring the ventricular anchor to the ventricle comprises using the ventricular anchor to anchor the tether to the ventricle so as to anchor the ventricular end of the net to the ventricle.

50. The method according to claim 37, wherein at least partially covering the atrial surface of the single native cardiac leaflet with the net comprises covering, with the net, only a portion of the atrial surface of single native cardiac leaflet, the portion including an annular edge of the single native cardiac leaflet.

51. The method according to claim 37, wherein at least partially covering the atrial surface of the single native cardiac leaflet with the net comprises covering, with the net, (a) a portion of the atrial surface of the single native cardiac leaflet, the portion including an annular edge of the single native cardiac leaflet, and (b) a free ventricular edge of the single native cardiac leaflet.

52. The method according to claim 37, wherein the net defines a plurality of openings and the net, when in a fully-expanded planar configuration, has an area, including the plurality of openings, of at least 100 mm2.

53. The method according to claim 37, wherein the net defines a plurality of openings having an average opening area of at least 4 mm2, when the net is in a fully-expanded planar configuration.

54. The method according to claim 53, wherein the average opening area is at least 10 mm2.

55. The method according to claim 37, wherein the net defines a plurality of openings, and wherein two or more of the openings have respective opening areas different from one another, when the net is in a fully-expanded planar configuration.

56. The method according to claim 55, wherein three or more of the openings have respective opening areas different from one another, when the net is in the fully-expanded planar configuration.

57. The method according to claim 56, wherein five or more of the openings have respective opening areas different from one another, when the net is in the fully-expanded planar configuration.

58. The method according to claim 55, wherein the opening area of a first one of the two or more of the openings equals at least 110% of the opening area of a second one of the two or more of the openings, when the net is in the fully-expanded planar configuration.

59. The method according to claim 58, wherein the opening area of the first opening equals at least 120% of the opening area of the second opening, when the net is in the fully-expanded planar configuration.

60. The method according to claim 55, wherein the opening areas of the plurality of openings vary along the net between the annular side and the ventricular end of the net.

61. The method according to claim 60, wherein the opening areas of the plurality of openings decrease along the net between the annular side and the ventricular end of the net.

62. The method according to claim 60, wherein the opening areas of the plurality of openings increase along the net between the annular side and the ventricular end of the net.

63. The method according to claim 55, wherein an average of the opening areas of the plurality of openings on an annular half of the net is different from an average of the opening areas of the plurality of openings on a ventricular half of the net.

64. The method according to claim 63, wherein the average of the opening areas of the plurality of openings on the annular half of the net is greater than the average of the opening areas of the plurality of openings on the ventricular half of the net.

65. The method according to claim 63, wherein the average of the opening areas of the plurality of openings on the annular half of the net is less than the average of the opening areas of the plurality of openings on the ventricular half of the net.

66. The method according to claim 37,
wherein the net comprises a plurality of flexible elongate members arranged so as to define a plurality of openings, and
wherein the net, when in a fully-expanded planar configuration, has a material surface area ratio of 5%-80%, the material surface area ratio defined as the ratio of material of the flexible elongate members to an area of the net, including the plurality of openings and the material of the flexible elongate members.

67. The method according to claim 37,
wherein the net comprises a plurality of flexible elongate members arranged so as to define a plurality of openings, and
wherein at least partially covering the atrial surface of the single native cardiac leaflet with the net comprises at least partially covering the atrial surface of the single native cardiac leaflet with the net such that a net surface area ratio is 5%-150%, the net surface area ratio being the ratio of an area of the net, including the openings and material of the net, to a total area of the atrial surface.

68. The method according to claim 37, wherein the net, when in a fully-expanded planar configuration, includes a triangular portion.

69. The method according to claim 37, wherein the net, when in a fully-expanded planar configuration, is triangular.

70. The method according to claim 37, wherein the net, when in a fully-expanded planar configuration, includes a rectangular portion.

71. The method according to claim 37, wherein the net, when in a fully-expanded planar configuration, is rectangular.

72. The method according to claim 37, wherein the net includes a plurality of flexible elongate members arranged at least partially as a grid when in a fully-expanded planar configuration.

73. The method according to claim 37, wherein the net includes a plurality of flexible elongate members arranged at least partially in a spider-web configuration when in a fully-expanded planar configuration.

74. The method according to claim 37,
wherein the leaflet restraint is a first leaflet restraint, the non-blood-occlusive net is a first non-blood-occlusive net, the two or more tissue-penetrating annulus anchors are two or more first tissue-penetrating annulus anchors, and the ventricular anchor is a first ventricular anchor, and
wherein the method further comprises:
anchoring two or more second tissue-penetrating annulus anchors of a second leaflet restraint to the annulus of the native atrioventricular valve along or within 1 cm of a portion of the annulus to which the single native cardiac leaflet attaches;
at least partially covering the atrial surface of the single native cardiac leaflet with a second non-blood-occlusive net of the second native leaflet restraint, wherein the second tissue-penetrating annular anchors are fixed to an annular side of the second net; and
anchoring a second ventricular anchor of the second leaflet restraint to the ventricle so as to anchor a ventricular end of the second net to the ventricle, such that the first and the second leaflet restraints limit leaflet with prolapse of the single native cardiac leaflet into the atrium.
